(19) United States
(12) Patent Application Publication
(10) Pub. No.: US 2013/0296569 A1
(43) Pub. Date: Nov. 7, 2013

(54) PROCESS FOR THE PREPARATION OF PHOSPHORIC ACID MONO-(1--4-METHOXYMETHYL-PIPERIDIN-4-YL) ESTER

(71) Applicants: Vijaykumar Jagdishwar Patil, Aurangabad (IN); Rajkumar Vishwanath Hangarge, Pune (IN); Burhanuddin Munshi Zaki Ahmed, Kalyan (IN); Loganathan Velupillai, Aurangabad (IN); Bharat Kalidas Trivedi, Farmington Hills, MI (US)

(72) Inventors: Vijaykumar Jagdishwar Patil, Aurangabad (IN); Rajkumar Vishwanath Hangarge, Pune (IN); Burhanuddin Munshi Zaki Ahmed, Kalyan (IN); Loganathan Velupillai, Aurangabad (IN); Bharat Kalidas Trivedi, Farmington Hills, MI (US)

(21) Appl. No.: 13/882,171

(22) PCT Filed: Feb. 3, 2011
(86) PCT No.: PCT/IB11/50460

§ 371 Date: Jul. 26, 2013

(30) Foreign Application Priority Data
Nov. 3, 2010 (IN) 3049/MUM/2010

(51) Int. Cl. C07F 9/6558; C07D 211/48; C07D 413/10; C07D 491/107

(52) U.S. Cl. CPC C07F 9/65583; C07D 491/107; C07D 211/48; C07D 413/10
USPC 546/16; 546/25; 546/216; 546/221; 546/209

(57) ABSTRACT

The invention relates to a process to prepare pharmacologically active phosphoric acid mono-(1-{4-[(S)-5-(acetylamino-methyl)-2-oxo-oxazolidin-3-yl]-2,6-difluoro phenyl}-4-methoxy methyl-piperidin-4-yl)ester.

### FIELD OF THE INVENTION

The invention relates to a process to prepare pharmacologically active Phosphoric acid mono-(1-{4-[(S)-5-(acetylamino-methyl)-2-oxo-oxazolidin-3-yl]-2,6-difluorophenyl}-4-methoxy methyl-piperidin-4-yl)ester.

### BACKGROUND OF THE INVENTION

Oxazolidinones represent a novel chemical class of synthetic antimicrobial agents. Linezolid represents the first member of this class to be used clinically. Oxazolidinones display activity against important Gram-positive human and veterinary pathogens including Methicillin-Resistant Staphylococcus aureus (MRSA), Vancomycin Resistant Enterococci (VRE) and β-lactam Resistant Streptococcus pneumoniae (PRSP). The oxazolidinones also show activity against Gram-negative aerobic bacteria, Gram-positive and Gram-negative anaerobes. (Diekema D J et al., Lancet 2001; 358: 1975-82).

Various oxazolidinones and their methods of preparation are disclosed in the literature. International (PCT) publication No. WO 1995/25106 discloses substituted piperidino phenyloxazolidinones and WO 1996/13502 discloses phenyloxazolidinones having a multisubstituted azetidinyl or pyrrolidinyl moiety. U.S. Patent application 2004/0063954, International (PCT) publication Nos. WO 2004/007489 and WO 2004/007488 disclose piperidinyl phenyl oxazolidinones for antimicrobial use. Pyrrolidinyl/piperidinyl phenyl oxazolidinone antibacterial agents are also described in Kim H Y et al., Bioorg. & Med. Chem. Lett., (2003), 13:2227-2230. International (PCT) publication WO 1996/35691 discloses spirocyclic and bicyclic diazinyl and carbazinyl oxazolidinone derivatives. Diazepeno phenyloxazolidinone derivatives are disclosed in the International (PCT) publication WO 1999/24428. International (PCT) publication WO 2002/06278 discloses substituted aminopiperidino phenyloxazolidinone derivatives.

Various other methods of preparation of oxazolidinones are reported in U.S. Pat. No. 7,087,784, U.S. Pat. No. 6,740,754, U.S. Pat. No. 4,948,801, U.S. Pat. No. 3,654,298, U.S. Pat. No. 5,837,870, Canadian Patent No. 681,830, J. Med. Chem., 32, 1673 (1989), Tetrahedron, 45, 1323 (1989), J. Med. Chem., 33, 2569 (1990), Tetrahedron Letters, 37, 7937-40 (1996) and Organic Process Research and Development, 11, 739-741 (2007).

The invention provides a process for the preparation of phosphoric acid mono-(1-[4-[(S)-5-(acetylamino-methyl)-2-oxo-oxazolidin-3-yl]-2,6-difluorophenyl]-4-methoxymethyl-piperidin-4-yl)ester, which is convenient and industrially applicable.

### SUMMARY OF THE INVENTION

The invention provides a novel process to prepare phosphoric acid mono-(1-{4-[(S)-5-(acetylamino-methyl)-2-oxo-oxazolidin-3-yl]-2,6-difluorophenyl}-4-methoxymethyl-piperidin-4-yl)ester of Formula (A).

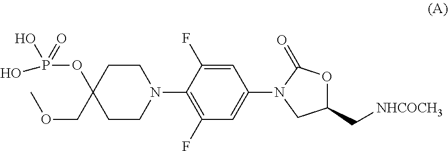

Other aspects will be set forth in the description which follows, and in part will be apparent from the description or may be learnt by the practice of the invention. The details of one or more embodiments of the inventions are set forth in the description below. Other features, objects and advantages of the inventions will be apparent from the description and claims.

### DETAILED DESCRIPTION OF THE INVENTION

The invention provides a process of preparation of phosphoric acid mono-(1-{4-[(S)-5-(acetylamino-methyl)-2-oxo-oxazolidin-3-yl]-2,6-difluorophenyl}-4-methoxymethyl-piperidin-4-yl)ester Formula (A) and various intermediates used in the process thereof.

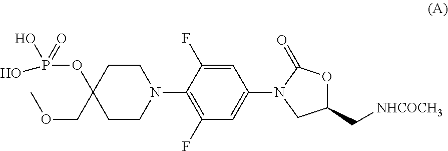

There is provided process for preparing compound having the structure (A)

The starting materials for producing intermediate of Formula (1) may be prepared by any of the methods known in the art such as U.S. Pat. No. 5,668,286; US Publication Nos. 2004/0063954 and 2005/0143421 or by procedures that would be well known to one of ordinary skill in the art of synthetic organic chemistry.

The following abbreviations are used in the text: DMF for N,N-dimethylformamide, DMSO for dimethyl sulfoxide, THF for tetrahydrofuran, ACN for acetonitrile, Ac₂O for acetic anhydride, LDA for lithium diisopropylamine, DMAc for dimethyl acetamide, CBZ-Cl for Benzylchloroformate, n-BuLi for n-Butyl Lithium, TLC for Thin Layer Chromatography, RT for Room Temperature, MP for Melting Point and MF for Molecular Formula.

The invention provides a process of preparation of compound of Formula (A) as depicted in scheme-1: which includes the steps of:

Converting intermediate of Formula (1) into epoxide intermediate of Formula (2) using oxyranylation reagents such as trimethyloxosulfonium iodide or trimethyloxosulfonium chloride in the presence of a base such as sodium hydride, potassium tert-butoxide, LDA, or n-butyllithium in a solvent such as DMSO, DMF, THF, ACN or a mixture thereof, at temperatures ranging from −10° C. to +100° C. The intermediate (2) is then treated with a suitable reagent like an alkoxide such as sodium methoxide or a base such as sodium carbonate, potassium carbonate, sodium tert-butoxide or potassium tert-butoxide in an alcoholic solvent such as methanol to yield the intermediate of Formula (3). The nitro group in intermediate of Formula (3) is reduced with a catalytic amount of reducing agent such as 10% Pd/C, platinum oxide, or Raney nickel, or Sodium dithionate, in various solvents such as methanol, ethyl acetate, acetone, acetonitrile at a temperature ranging from room temperature to reflux, to obtain the corresponding amino intermediate compound of Formula (4). The amino intermediate is further treated with benzyl chloroformate in presence of a base such as sodium carbonate, potassium carbonate or ammonia and a solvent like chloroform or dichloromethane, to give intermediate of Formula (5). The intermediate of Formula (5) is treated with R-(−)-glycidyl butyrate in the presence of a base such as n-butyl lithium, lithium diisopropylamine, lithium hexamethyldisilazane, lithium tert-butoxide, sodium amide and sodium hydride using a dry solvent like THF, DMF or DMSO at a temperature ranging from −78° to +75° C. to give the intermediate of Formula (6). The intermediate of Formula (6) is treated with methanesulphonyl chloride in the presence of a base such as triethylamine or pyridine using a solvent such as chloroform or dichloromethane to give the intermediate of Formula (7). The intermediate of Formula (7) is converted into intermediate of Formula (8) by treating intermediate (7) with sodium azide in a solvent such as DMSO, DMF or aqueous DMF or DMAc. Alternatively the intermediate (6) is treated with diphenylphosphoryl azide in the presence of base such as DBU using a solvent such as THF to give the intermediate of Formula (8a) where T is azide. By treating intermediate (7) with pthalamide salt such as potassium pthalamide or treating intermediate of Formula (7) with diformylamide to obtain the intermediate compound of Formula (8b) or (8c). The intermediate of Formula (8a) is converted into amino intermediate of Formula (9) using a catalyst such as 5% palladium on carbon, 10% palladium on carbon, 20% palladium hydroxide on carbon, platinum on carbon or Raney-Nickel in the presence of a hydrogen source such as hydrogen gas in a solvent such as methanol, ethanol, ethyl acetate, tetrahydrofuran, or a mixture thereof. Alternately, the intermediate of Formula (8a) can be reduced to amino compound by using the reagent sodium borohydride-cobalt chloride in a solvent such as tetrahydrofuran or by treating with triphenyl phosphine followed by water in a suitable solvent and isolating the free amine. The amino compound of Formula (9) is further treated with a suitable reagent such as acetic anhydride in the presence of a base such as triethylamine or pyridine in an organic solvent such as chloroform, dichloromethane, ethylacetate, to give the corresponding acetamide intermediate of Formula (10). The acetamide intermediate of Formula (10) is further phosphorylated with a suitable phosphorylating reagent like phosphorous trichloride or a phosphoramidite like dibenzyl-N,N,diisopropylphosphoramidite in the presence of a suitable coupling reagent like tetrazole and the like to obtain the intermediate of Formula (11). The intermediate (11) is further converted into the compound of Formula (A) by carrying out debenzylation with 5-10% Pd/C in a suitable solvent like methanol, ethyl acetate, acetone etc.

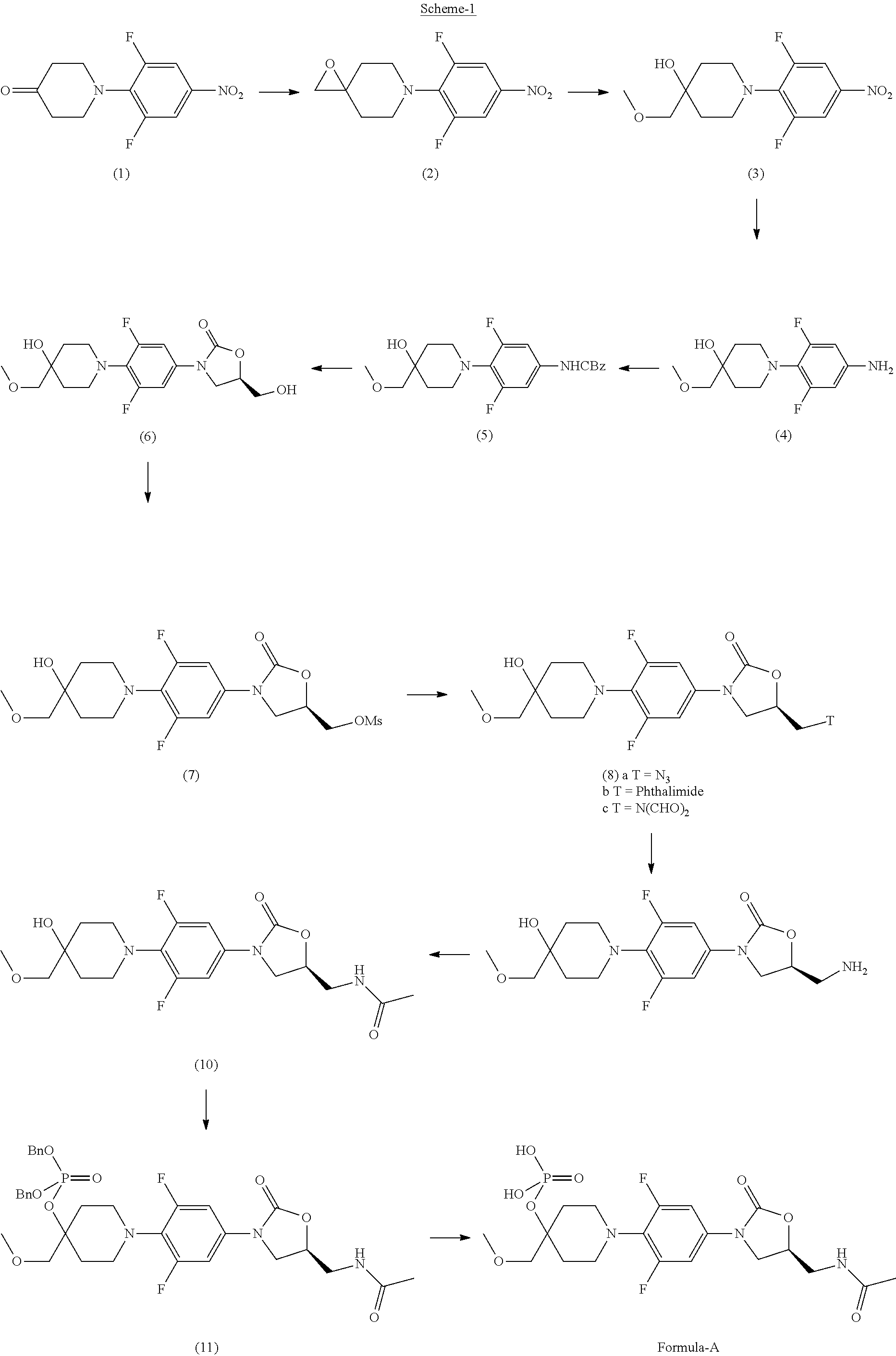

wherein T is azide, or pthalimide or diformylamino.

In an embodiment of the invention is to provide a novel method of preparation of the compound of Formula (3), which includes the steps of:

Converting intermediate of Formula (1) directly into intermediate of Formula (3) by adding intermediate (1) in small slots to a previously stirred (30 minutes) and cooled (10° C.-15° C.) solution mixture of Dimethylsulfoxide, an alcoholic solvent like methanol, a base such as potassium hydroxide or sodium methoxide and an oxyranylation reagent such as trimethylsulfoxonium iodide followed by further stirring for 24 hours at RT (where ring opening of the epoxide intermediate viz. 6-(2,6-difluoro-4-nitrophenyl)-1-oxa-6-azaspiro[2.5]octane takes place).

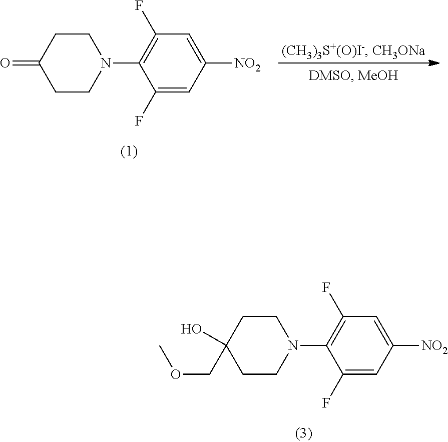

In an embodiment of the invention is to provide a novel method of preparation of the compound of Formula (5), which includes the steps of:

Converting intermediate of Formula (3) into intermediate of Formula (5) by heating a solution of (3) in aqueous alcohol like aqueous methanol with a reducing agent such as Sodium dithionite at 80° C. for 6-10 hours (wherein a reaction mixture containing the intermediate 1-(4-amino-2,6-difluoro-phenyl)-4-methoxymethyl-piperidin-4-ol is produced) and recovering methanol under vacuum below 65° C. and extracting aqueous residue with chloroform that is dried over anhydrous sodium sulfate. This chloroform extract on stirring with Benzylchloroformate solution (50% in toluene) at 15° C.-20° C. for 2-4 hours with a base such as sodium bicarbonate, potassium bicarbonate and the like, provide intermediate of Formula (5). Alternatively intermediate (3) can be hydrogenated over 10% Pd—C, in a solvent like ethyl acetate, at 30 psi, at temperatures between 25-80° C., for 3-6 h. The catalyst is filtered and the filtrate on stirring with Benzylchloroformate solution (50% in toluene) at 15° C.-20° C. for 2-4 hr with a base such as sodium bicarbonate, potassium bicarbonate and the like, provides intermediate of Formula (5).

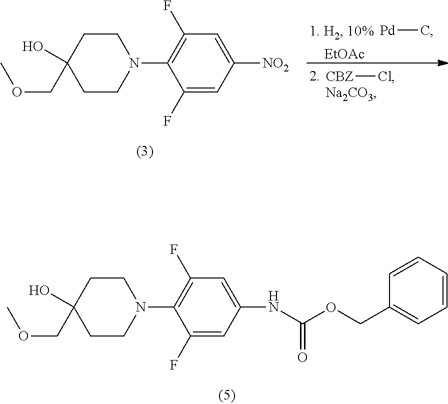

Yet another embodiment of the invention is to provide methods of preparation of the compound of Formula (6) that includes the steps of:

Converting intermediate of Formula (5) into intermediate of structure (6) by stirring a solution of intermediate of Formula (5) in a mixture of a base such as n-BuLi, lithium diisopropylamine, lithium hexamethyldisilazane, lithium tertbutoxide, sodium amide and sodium hydride using a dry solvent like THF, DMF or DMSO (mixture being previously stirred for 1 h at 40° C.) with R-(−)-glycidyl butyrate for 5-6 h at 40° C.

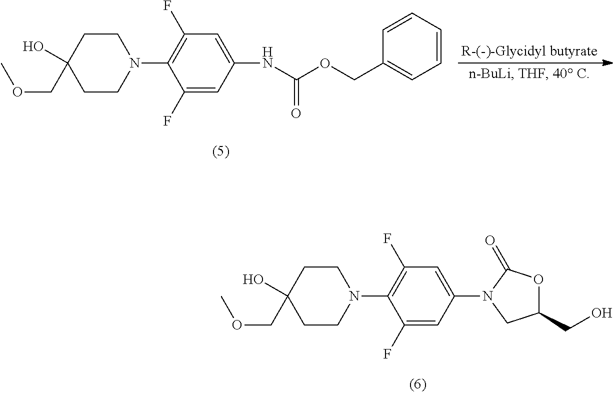 [SMILES: COCC1(O)CCN(c2c(F)cc(NC(=O)OCc3ccccc3)cc2F)CC1 ; "R-(-)-Glycidyl butyrate" ; "n-BuLi, THF, 40° C." ; COCC1(O)CCN(c2c(F)cc(N3C[C@H](CO)OC3=O)cc2F)CC1]

Yet another embodiment of the invention is to provide methods of preparation of the compound of Formula (10) that includes the steps of:

Converting intermediate of Formula (6) into intermediate of Formula (10) by stirring a solution of intermediate of Formula (6) in a mixture of acetamide, triphenylphosphine, an azo compound such as diethyldiazocarboxylate, diisopropyl azo dicarboxylate and the like using a solvent such as tetrahydrofuran, dimethylformamide, dimethylsulfoxide and the like for 10-20 hours at room temperature.

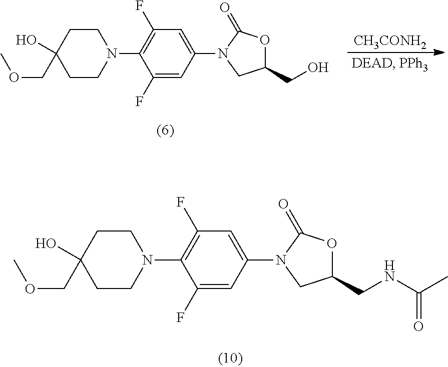 [SMILES: COCC1(O)CCN(c2c(F)cc(N3C[C@H](CO)OC3=O)cc2F)CC1 ; "CH3CONH2" ; "DEAD, PPh3" ; COCC1(O)CCN(c2c(F)cc(N3C[C@H](CNC(C)=O)OC3=O)cc2F)CC1]

Alternatively intermediate of Formula (6) can be converted into intermediate of Formula (8) by stirring a solution of intermediate of Formula (6) in a mixture of phthalimide, triphenylphosphine, an azo compound such as diethyldiazocarboxylate, diisopropyl azo dicarboxylate and the like using a solvent such as tetrahydrofuran, dimethylformamide, dimethylsulfoxide and the like for 5-15 h at room temperature.

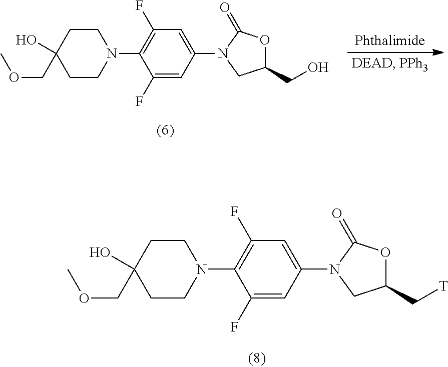

Alternatively, converting intermediate of Formula (6) into intermediate of Formula (8) by stirring a solution of intermediate of Formula (6) containing Diphenyl phosphoryl azide with

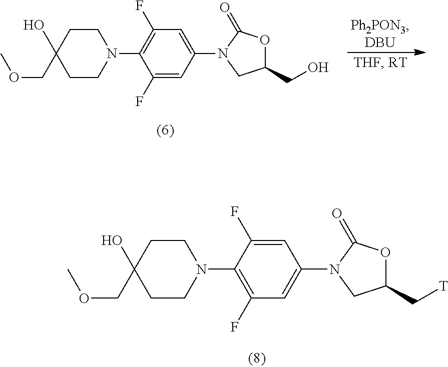

DBU using a solvent such as tetrahydrofuran, 1,4-dioxane, di-isopropyl ether and the like for 5-15 h at room temperature.

Converting intermediate of Formula (8) into intermediate of Formula (10) by stirring a solution of intermediate of Formula (8) with hydrazine hydrate in a solvent such as methanol, ethanol, isopropyl alcohol, butanol and the like for 4-8 hours at room temperature. The solvent is evaporated to obtain a residue that is treated with 3% sodium carbonate and extracted with a halogenated solvent such as dichloromethane, chloroform, carbontetrachloride and the like. The organic layer is dried and is stirred with an acetylating agent such as acetic anhydride, acetyl chloride and the like in presence of a base such as triethylamine, pyridine, ammonia, ammonium hydroxide and the like for 4-8 hours at room temperature.

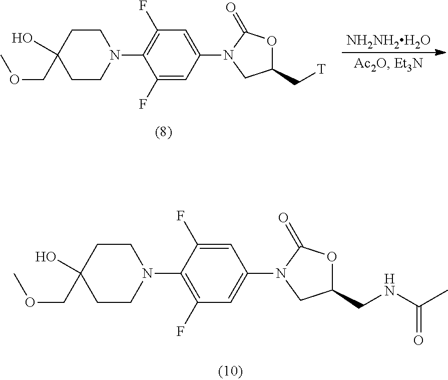

Yet another embodiment of the invention is to provide methods of preparation of the intermediate of Formula (10) that includes the steps of:

Converting intermediate of Formula (7) into intermediate of Formula (10) by stirring intermediate of Formula (7) with sodium diformylamide in a solvent such as dimethylformamide, dimethylsulfoxide and the like for 10-20 h at a temperature of 75-125° C. To this reaction mixture, a mixture of an acid such as conc. HCl, water and a solvent such as methanol, ethanol, isopropyl alcohol, butanol and the like is added and reaction mixture is further stirred for 4-8 h at a temperature of 60-90° C. The mixture is concentrated under reduced pressure at 60-75° C. The resulting mixture is further stirred with a mixture of water, a base such as ammonia, triethylamine, pyridine, ammonium hydroxide and the like and an acetylating agent such as acetic anhydride and acetyl chloride at 25-45° C. for 3-6 hrs.

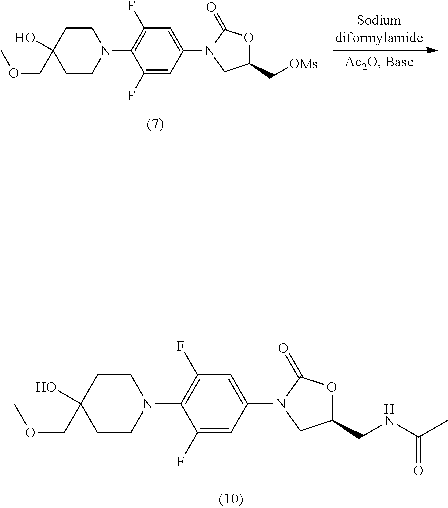 [SMILES: COCC1(O)CCN(c2c(F)cc(N3C[C@H](COS(C)(=O)=O)OC3=O)cc2F)CC1 ; COCC1(O)CCN(c2c(F)cc(N3C[C@H](CNC(C)=O)OC3=O)cc2F)CC1]

Yet another embodiment of the invention is to provide methods of preparation of the compound of Formula (A) that includes the steps of:

Converting intermediate of Formula (10) into intermediate of Formula (11), by stirring intermediate of Formula (10) with a suitable phosphorylating reagent such as phosphorous trichloride or a phosphoramidite like dibenzyl-N,N,diisopropylphosphoramidite., in the presence of an activating agent such as tetrazole, trimethyl silyl chloride, pyridinium hydrochloride, pyridinium trifluoroacetate, 4,5-dicyanoimidazole, pyridinium trifluomethanesulfonate, pyridinium acetate, pyridinium chloroacetate, pyridinium dichloro acetate, polyvinyl pyridinium hydrochloride, 2-amino-4,6-dimethylpyrimidinium trifluoroacetate, imidazolium hydrochloride, imidazolium trifluoroacetate, aniline hydrochloride, p-anisidine trifluoroacetate, o-toluidine hydrochloride, p-toluidine hydrochloride or phenanthrene trifluoroacetate, in a solvent such as dichloromethane, chloroform, carbon tetrachloride and the like for 2-6 hr. The resulting mixture is cooled and a solution of an oxidizing agent such as hydrogen peroxide (30%, 50% or 90%), urea hydrogen peroxide, peracetic acid, per trifluoroacetic acid, iodobenzene diacetate, m-chloroperbenzoic acid or mixtures thereof in dichloromethane is added. After 2-6 hours the solvent is evaporated under residue pressure and the residue is chromatographed.

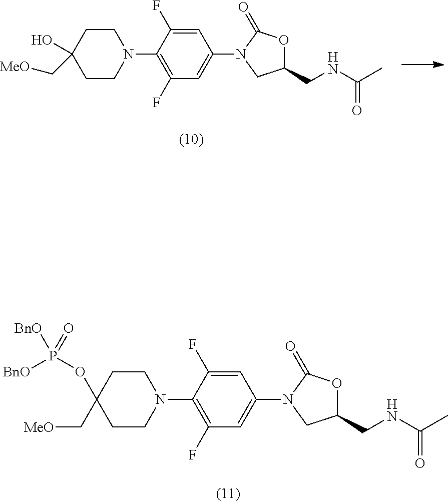 [SMILES: COCC1(O)CCN(c2c(F)cc(N3C[C@H](CNC(C)=O)OC3=O)cc2F)CC1 ; COCC1(OP(=O)(OCc2ccccc2)OCc2ccccc2)CCN(c2c(F)cc(N3C[C@H](CNC(C)=O)OC3=O)cc2F)CC1]

Yet another embodiment of the invention is to provide methods of preparation of the compound of Formula (A) that includes the steps of:

Converting intermediate of Formula (11) into compound of Formula (A) or pharmaceutically acceptable salts thereof, by stirring a suspension of compound of Formula (11) and a catalyst such as 20% palladium hydroxide in a solvent such as a mixture of dichloromethane/aqueous methanol for 4-8 hr.

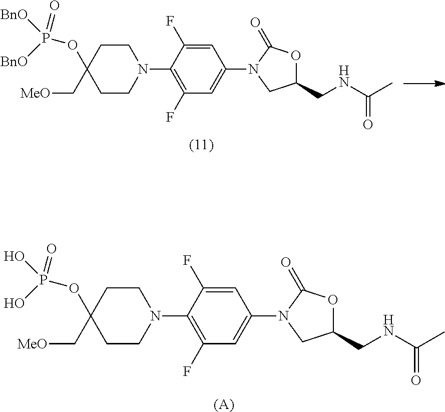

Specific intermediate compounds of the invention include:
6-(2,6-difluoro-4-nitrophenyl)-1-oxa-6-azaspiro[2.5]octane;
1-(2,6-Difluoro-4-nitro-phenyl)-4-methoxymethyl-piperidin-4-ol;
[3,5-Difluoro-4-(4-hydroxy-4-methoxymethyl-piperidin-1-yl)-phenyl]-carbamic acid benzyl ester;
(5R)-3-[3,5-Difluoro-4-(4-hydroxy-4-methoxymethyl-piperidin-1-yl)-phenyl]-5-hydroxymethyl-oxazolidin-2-one;
(5R)-Methanesulfonic acid 3-[3,5-difluoro-4-(4-hydroxy-4-methoxymethyl-piperidin-1-yl)-phenyl]-2-oxo-oxazolidin-5-ylmethyl ester;
(5R)-3-[3,5-Difluoro-4-(4-hydroxy-4-methoxymethyl-piperidin-1-yl)-phenyl]-5-azidomethyl-oxazolidin-2-one; and
(5S)-N-{3-[3,5-Difluoro-4-(4-hydroxy-4-methoxymethyl-piperidin-1-yl)-phenyl]-2-oxo-oxazolidin-5-ylmethyl}-acetamide.

Certain modifications and equivalents will be apparent to those skilled in the art and are intended to be included within the scope of the invention.

### EXAMPLES

### Preparation of Intermediate-1: 1-(2,6-Difluoro-4-nitrophenyl)-piperidin-4-one

Chloroform (9.3 L) was charged in a 20 L reaction assembly and 4-piperidone hydrochloride (1.17 Kg, 7.62 mol) was added under stirring followed by triethylamine (2.14 Kg, 2.95 L, 21.1 mol). After 30 minutes of stirring, 3,4,5-trifluoronitrobenzene (1.5 Kg, 8.47 mol) was added to the mixture in one lot and the contents were heated to 65-70° C. for 8 h. After completion of the reaction, chloroform was removed under vacuum to obtain a syrupy mass. At this stage, water (10 L) was added to the mass and the chloroform recovery was continued under vacuum below 65° C. till the chloroform was removed completely. The slurry was cooled to RT and filtered. The solid product was washed with water (3 L) followed by hexanes (2 L). The product was dried in a vacuum oven below 70° C. to obtain the product as a yellow solid, 1.88 Kg; Yield 97%.

M.P.: 130-132° C.; MS: 257(M+1); M.F.: C₁₁H₁₀F₂N₂O₃.

### Preparation of Intermediate 3: 1-(2,6-Difluoro-4-nitro-phenyl)-4-methoxymethyl-piperidin-4-ol

### Method A

### Preparation of Intermediate-2: (Stage-I): 6-(2,6-difluoro-4-nitrophenyl)-1-oxa-6-azaspiro[2.5]octane

A solution of trimethylsulfoxonium iodide (1.504 kg, 6.836 mol) in acetonitrile (7 L) was cooled to 0 to 5° C., under argon atmosphere. Potassium tert-butoxide (0.736 kg, 6.552 mol) was added in small lots over 0.5 h. The resulting solution was stirred for 2 h at the same temperature. To this solution was added 1-(2,6-Difluoro-4-nitrophenyl)-piperidin-4-one (1.4 kg, 5.46 mol) in small lots over a period of 1 h, while maintaining the temp. between 5-10° C. The resulting mixture was stirred for 1 h. The solvent was evaporated to a minimum amount possible, under reduced pressure while maintaining the temperature below 10° C. The residue was poured in water (18 L) and the pH adjusted to neutral with dilute acetic acid. The resulting slurry was stirred well and the separated solid filtered under suction. The solid was washed with fresh water till the filtrate was free of acetic acid. The solid was dried at 80° C., for 6 h, under reduced pressure to obtain the product as pale yellow solid, 1.264 kgs, yield 85%.

M.P.: 96-97° C.; MS: M+1: 271; M.F.: C₁₂H₁₂F₂N₂O₃.

### Preparation of Intermediate-3: (Stage-II): 1-(2,6-Difluoro-4-nitro-phenyl)-4-methoxymethyl-piperidin-4-ol

To a solution of sodium methoxide (236 g, 4.35 mol) in methanol (3 L), at RT, was added 6-(2,6-difluoro-4-nitrophenyl)-1-oxa-6-azaspiro[2.5]octane (964 g, 3.57 mol) in small portions and the reaction mixture was stirred for 26 h at RT. Acetic acid (265 g, 4.44 mol) was added slowly to neutralize the pH of the solution. The resulting mixture was poured into chilled water (18 L) and stirred for 1 h. The separated solid was filtered under suction. The solid was washed with additional water till the filtrate was free of acetic acid. The solid was dried for 10hat RT under reduced pressure, to obtain the product as a pale yellow solid, 973 g, yield, 90%

M.P.: 84-86° C.; MS: 303 (M+1); M.F.: C₁₃H₁₆F₂N₂O₄

### Method B

Dimethylsulfoxide (DMSO, 100 ml) and methanol (500 ml) were charged in a 1 L glass reaction assembly. Potassium hydroxide (59.2 g, 0.898 mol) was charged in the assembly followed by trimethylsulfoxonium iodide (94.5 g, 0.43 mol) and the contents were stirred for 30 minutes and then cooled to 10° C.-15° C. To the cooled contents was added 1-(2,6-difluoro-4-nitrophenyl)-piperidin-4-one (100 g, 0.39 mol) in small lots. After the addition, the temperature was allowed to raise to RT and the contents were further stirred for 24 h (ring opening of the epoxide intermediate viz. 6-(2,6-difluoro-4-nitrophenyl)-1-oxa-6-azaspiro[2.5]octane takes place).

[Physical data of the intermediate: M.P.: 96-97° C., MS: 271(M+1); M.F.: C₁₂H₁₂F₂N₂O₃.

After completion of the reaction the contents were poured slowly in ice-water (600 g crushed ice in 600 ml water). The precipitated solid product was filtered and was washed with water:methanol, 2:1 (100 ml×2). The wet product was used in the next step.

M.P.: 84-86° C.; MS: 303 (M+1); M.F.: C₁₃H₁₆F₂N₂O₄:

### Preparation of Intermediate-5: [3,5-Difluoro-4-(4-hydroxy-4-methoxymethyl-piperidin-1-yl)-phenyl]-carbamic acid benzyl ester

### Method A

### Preparation of Intermediate 4: (Stage-I)

Water (1.19 L) and methanol (595 nil) were charged in a 3 L glass reaction assembly, followed by 1-(2,6-difluoro-4-nitro-phenyl)-4-methoxymethyl-piperidin-4-ol (85 g, 0.281 mol) and the contents were stirred. Sodium dithionite (288 g, 1.407 mol) was added in one lot and the reaction mixture was heated to 80° C. for 8 h. After completion of the reaction (TLC), methanol was recovered under vacuum below 65° C. After the recovery, the aqueous residue was extracted with chloroform (400 ml×3). The combined chloroform extract (containing the intermediate 1-(4-amino-2,6-difluoro-phenyl)-4-methoxymethyl-piperidin-4-ol) was dried over anhydrous Sodium sulfate and used in the next step (carbamate formation).

### Preparation of Intermediate-5

### Stage-II

The above chloroform extract was charged in a 3 L glass reaction assembly. Sodium bicarbonate (70 g, 0.843 mol) was added to the extract and the contents were cooled to 15° C.-20° C. Benzylchloroformate solution (50% in toluene, 48 g, 96 ml, 0.281 mol) was added slowly to the above mixture under stirring. After completion of the addition, the reaction mixture was stirred at RT for 2 h. After completion of the reaction (TLC), the contents were filtered on a Buchner assembly and the solid cake was washed with chloroform (85 ml×2). The combined filtrate was evaporated under vacuum below 50° C. to obtain yellowish oily mass, which was poured slowly in hexanes (850 ml) under stirring to obtain a precipitate. The precipitated product was filtered and washed with hexanes (100 ml×2). The product was dried in a vacuum oven below 65° C. to obtain 60.2 g brownish product (Yield=38% on the basis of step-I input).

M.P.: 138-140° C.; MS: 407(M+1); M.F.: C₂₁H₂₄F₂N₂O₄.

### Method B

### Preparation of Intermediate 4: (Stage-I):

To a solution of 1-(2,6-difluoro-4-nitro-phenyl)-4-methoxymethyl-piperidin-4-ol (973 g, 3.22 mol) in ethyl acetate (10 L) was added 10% Pd—C, (250 g, 50% wet) and the resulting miture was hydrogenated in a pressure at 30 PSI, 45-55° C., for 3 h. The catalyst was filtered and the residue was washed with additional ethyl acetate (200 ml). The combined filtrates were used as such for the next reaction (carbamate formation)

### Preparation of Intermediate-5

### Stage-II

To the above filtrate was added sodium bicarbonate (406 g, 4.83 mol) and the mixture warmed to 40-45° C. To this mixture was added a 50% solution of Benzyl chloroformate in toluene (1.373 L, 4.025 mol), drop-wise, over a period of 1 h. Stir the resulting mixture for 1 h and filter the insoluble material. The residue was washed with 300 ml of ethyl acetate. The filtrates were combined and the solvent evaporated under reduced pressure, below 55° C. Cool the residue and dilute it with hexane (10 L). The resulting slurry was stirred well and the separated solid was filtered under suction. The residue was washed with additional hexane (2 L). The solid was dried for 10 h at RT, to obtain the product as dark brown solid, 1200 g, yield, 96%.

M.P.: 138-140° C.; MS: 407(M+1); M.F.: C₂₁H₂₄F₂N₂O

### Preparation of Intermediate-6: (5R)-3-[3,5-Difluoro-4-(4-hydroxy-4-methoxymethyl-piperidin-1-yl)-phenyl]-5-hydroxymethyl-oxazolidin-2-one

To a mixture of [3,5-difluoro-4-(4-hydroxy-4-methoxymethyl-piperidin-1-yl)-phenyl]-carbamic acid benzyl ester (100 g, 0.237 mol) in dry tetrahydrofuran (THF) (2 L) at 40° C. was added drop-wise n-BuLi in hexane (1.6M, 45.5 g, 455 ml, 0.711 mol) under nitrogen atmosphere. The contents were stirred for 1 h at 40° C. and R-(−)-glycidyl butyrate (68.25 g, 0.474 mol) was added gradually. After the addition of R-(−)-glycidyl butyrate, the reaction mixture was stirred for 5-6 h at 40° C. till completion of the reaction (TLC). After completion of the reaction, a solution of sodium methoxide (2 g) in methanol (66 ml) was added to the contents followed by water (8 ml) and the contents were stirred for an additional 0.5 h. Water (1 L) was added to the solution and the contents were extracted with ethyl acetate (1 L). The aqueous layer was further extracted with ethyl acetate (3×500 ml). The combined organic layer was evaporated under vacuum to obtain a thick residue. tert-Butyl methyl ether (1 L) was added to the residue and the contents were stirred for about 1 h to obtain a solid product, which was filtered and washed with tert-butyl methyl ether (2×100 ml). The product was dried under vacuum below 60° C. to obtain the product as a 46.5 g dark brown compound, 46.5 g, yield 51%.

M.P.: 117-119° C.; MS: 373(M+1); M.F.: C₁₇H₂₂F₂N₂O₅.

### Preparation of Intermediate-7: (5R)-Methanesulfonic acid 3-[3,5-difluoro-4-(4-hydroxy-4-methoxymethyl-piperidin-1-yl)-phenyl]-2-oxo-oxazolidin-5-ylmethyl ester

To a mixture of (5R)-3-[3,5-difluoro-4-(4-hydroxy-4-methoxymethyl-piperidin-1-yl)-phenyl]-5-hydroxymethyl-oxazolidin-2-one (45 g, 0.121 mol) in dichloromethane (0.3 L), was added triethylamine (24.5 g, 34 ml, 0.242 mol) while stirring. Methanesulfonyl chloride (18 g, 12.2 ml, 0.157 mol) was added to the above solution over a period of 1 h at 10° C.-20° C. and the reaction mixture was stirred for additional 2 h at RT. After completion of the reaction (TLC), the contents were evaporated under vacuum at 40° C. to obtain an oily residue. Water (450 ml) was added to the residue and the traces of dichloromethane were removed under vacuum. The solid product thus obtained was filtered, washed with water (2×50 ml) and dried under vacuum at 70° C. to obtain 50.6 g brownish compound. Yield=93%;

M.P.:106-108° C.; MS: 451(M+1); M.F.: C₁₈H₂₄F₂N₂O₇S.

### Preparation of Intermediate 8a: (5R)-3-[3,5-Difluoro-4-(4-hydroxy-4-methoxymethyl-piperidin-1-yl)-phenyl]-5-azidomethyl-oxazolidin-2-one

### Method A

To a solution of (R)-3-(3,5-difluoro-4-(4-hydroxy-4-(methoxymethyl)piperidin-1-yl)phenyl)-5-(hydroxymethyl)oxazolidin-2-one (2 g, 5.3 mmol),in tetrahydrofuran (20 mL), under argon, was added diphenylphosphoryl azide (1.63 mL, 5.9 mmol). The solution was cooled to 0° C. in an ice-bath. 1,8-diazabicyclo[5.4.0]undec-7-ene (DBU) (0.76 mL, 4.9 mmol) was added drop-wise over 15 min. The reaction was stirred at same temperature for 1 hr, and then warmed to room temperature and stirred under for 16 hr. The reaction mixture was diluted with ethyl acetate (20 mL), and water (20 mL). After separation of water layer, the organic layer was washed with water and 0.5M citric acid monohydrate (10 mL). The organic layer was dried over sodium sulfate and the solvent evaporated under reduced pressure. The residue was triturated with ether to obtain the product as a buff colored solid, 1.32 g (62%).

M.P.: 106-108° C.; M.S.—398(M+1); M.F.—C₁₇H₂₁F₂N₅O₄,

### Method B

To a solution of (5R)-methanesulfonic acid 3-[3,5-difluoro-4-(4-hydroxy-4-methoxymethyl-piperidin-1-yl)-phenyl]-2-oxo-oxazolidin-5-ylmethyl ester (20 g, 0.044 mol, wet) in N,N-dimethylformamide (30 ml), was added sodium azide (8.6 g, 0.133 mol) in a single lot. The reaction mixture was gradually heated and the temperature was maintained at 70° C. for 8 h. After completion of the reaction (TLC), the contents were cooled to 20-25° C. and poured slowly into chilled water (300 ml). The solid product thus obtained was filtered and washed with water (2×50 ml). The wet product was air dried to obtain 16.5 g dark brown compound (being an azide, it was NOT exposed to heat during drying) Yield ˜93%.

M.P.: 106-108° C.; MS: 398(M+1); M.F.: C₁₇H₂₁F₂N₅O₄;

### Preparation of Intermediate 8b: (5S)-N-2-{3-[3,5-Difluoro-4-(4-hydroxy-4-methoxymethyl-piperidin-1-yl)-phenyl]-2-oxo-oxazolidin-5-ylmethyl}-phthalimide

### Method A

A mixture of (5R)-{3-[3,5-Difluoro-4-(4-hydroxy-4-methoxymethyl-piperidin-1-yl)phenyl]-2-oxo-oxazolidin-5-yl methyl}-methanesulfonate (10 g, 0.022 mol), Potassium phthalimide (12.2 g, 0.066 mol) and DMF (50 ml) was heated, with stirring, at 90° C. for 4 h. The resulting mixture was cooled to RT and poured over ice-water mixture. The separated solid was filtered, washed with water and dried under suction to obtain the product as a white solid, 9.46 g, in 85% yield.

M.P.: 154-156° C.; MS: 502 (M+1); M.F. C₂₅H₂₅F₂N₃O₆

### Method B

To tetrahydrofuran (30 ml) were added triphenylphosphine (2.11 g, 8 mmol)) and diethyldiazocarboxylate (1.62 g, 8 mmol)), and the solution stirred at room temperature. After 10 minute phthalimide (1.18 g, 8 mmol)) was added and after a further stirring for 10 minute, (R)-3-(3,5-difluoro-4-(4-hydroxy-4-(methoxymethyl)piperidin-1-yl)phenyl)-5-(hydroxymethyl)oxazolidin-2-one (2 g, 5.3 mmol) was added and stirring continued further at room temperature. After 8 hrs ice-cold water (4 ml) was added to the reaction mixture and the resulting mixture was extracted by ethyl acetate (2×20 ml). The ethyl acetate extract was dried (over sodium sulfate) and concentrated under reduced pressure. The residue was chromatographed on a column of silica gel to obtain the product as an off-white solid, 1.56 g, yield 58%.

M.P.: 154-156° C.; MS: 502 (M+1); M.F. C₂₅H₂₅F₂N₃O₆.

### Preparation of Intermediate 10: (5S)-N-{3-[3,5-Difluoro-4-(4-hydroxy-4-methoxymethyl-piperidin-1-yl)-phenyl]-2-oxo-oxazolidin-5-ylmethyl}-acetamide via

### Intermediate 9: 5-aminomethyl-3-[3,5-difluoro-4-(4-hydroxy-4-methoxymethyl-piperidin-1-yl)-phenyl]-oxazolidin-2-one

### Method A

To a solution of (5R)-{3-[3,5-difluoro-4-(4-hydroxy-4-methoxymethyl-piperidin-1-yl)-phenyl]-5-azidomethyl-oxazolidin-2-one (10 g, 0.025 mol) in methanol (100 ml), were charged cobalt chloride (0.6 g, 0.0025 mol) followed by sodium borohydride (0.95 g, 0.025 mol) in small lots over a period of 30 minutes. The reaction mixture was stirred at RT for additional 2 h. After completion of the reaction, the contents were evaporated under vacuum below 40° C. to obtain a sticky mass. The contents were suspended in a mixture of water (100 ml) and ethyl acetate (50 ml) and stirred for 15 minutes. The contents were filtered through a filter-aid bed and the bed was washed with ethyl acetate (2×25 ml). The layers were separated and the aqueous layer was further extracted with ethyl acetate (4×50 ml). The combined organic layer was washed with 1% HCl solution (100 ml). The aqueous layer was separated and washed with dichloromethane (4×50 ml). The pH of the aqueous layer was adjusted to 8 by adding saturated sodium bicarbonate solution. The contents were extracted with ethyl acetate (6×50 ml) till no amine spot was seen in the final organic extract. The combined organic layer (containing the intermediate 5-aminomethyl-3-[3,5-difluoro-4-(4-hydroxy-4-methoxymethyl-piperidin-1-yl)-phenyl]-oxazolidin-2-one) was dried over anhydrous sodium sulfate.

Triethylamine (3.3 g, 4.5 ml, 0.0327 mol) was added to the above organic layer and acetyl chloride (2.17 g, 2 ml, 0.0277 mol) was added gradually over a period of 1 h at RT. The reaction mixture was stirred for 2 h and after completion of the reaction (TLC), the contents were washed with water (50 ml) and the layers separated. Activated carbon (1 g) was added to the organic layer and the contents were stirred for 15 minutes. The contents were filtered on a celite bed and the carbon-celite bed was washed with ethyl acetate (2×10 ml). The combined filtrate was evaporated under vacuum to obtain a slurry, which was filtered on a Buchner assembly and the product was washed with ethyl acetate (2×10 ml). The product was dried under vacuum at 70° C. to obtain 5 g off-white solid. Yield=48% (on the basis of azide). HPLC Purity ˜98%.

M.P.: 178-179° C.; MS: 414 (M+1); M.F.: C₁₉H₂₅F₂N₃O₅.

### Method B

A solution of (5R)-3-[3,5-difluoro-4-(4-hydroxy-4-methoxymethyl-piperidin-1-yl)-phenyl]-5-azidomethyl-oxazolidin-2-one (50 g, 0.125 mol) in ethyl acetatel (1 L ml), were charged with 5 g of 10% of Pd—C catalyst (50% wet) and the resulting mixture was hydrogenated at 30 psi for 3 h at 50° C. The resulting mixture was cooled and filtered under suction over celite bed. The residue was washed with additional ethyl acetate (200 ml). The combined filtrates were concentrated to 500 ml volume.

To the above ethyl acetate solution was added Triethyl amine (19.1 g, 0.189 mol), and acetic anhydride (16.1 g, 1.58 mol) in a single lot in few minutes). The reaction mixture was stirred for 16 h at R.T. The resulting mixture was cooled to 0-5° C., stirred for 0.5 h and filtered under suction. The residue was washed with cold ethyl acetate (100 ml) and dried at 70° C. under reduced pressure to obtain the product as a a off-white solid, 43.5 g, in 84% yield over two steps.

HPLC Purity ˜98%

M.P.: 178-179° C.; MS: 414 (M+1); M.F.: C₁₉H₂₅F₂N₃O₅.

### Method C

To a solution of (S)-N-2-{3-[3,5-Difluoro-4-(4-methoxymethyl-4-hydroxypiperidine-1yl)phenyl]-2-oxo-oxazolidin-5-yl methyl}-phthalimide (2.77 g, 0.0055 mol) in ethanol (20 ml) was added hydrazine hydrate (0.554 g, 0.011 mol) and the resulting solution stirred at RT for 6 h. The solvent was evaporated under reduced pressure, the residue suspended in 3% sodium carbonate solution and extracted in dichloromethane (40 ml). The dichloromethane layer was dried and to this solution was added triethylamine (1.11 g, 0.011 mol) and acetic anhydride (0.67 g, 0.007 mol) and the solution stirred for 6 h at RT. The solvent was evaporated under reduced pressure and the residue purified by flash chromatography to obtain the product as white solid, 1.94 g, in 85% yield.

M.P.: 178-179° C.; MS: 414 (M+1); M.F.: C₁₉H₂₅F₂N₃O₅.

### Method D

A mixture of (5R)-{3-[3,5-Difluoro-4-(4-hydroxy-4-methoxymethyl-piperidin-1-yl)phenyl]-2-oxo-oxazolidin-5-yl methyl}-methanesulfonate (1 μm, 4.4 mmol) and sodium diformylamide (2 gms, 22 mmol) in DMF (5 ml) was stirred at 95° C. for 15 hrs. Then a mixture of conc. HCl (0.6 ml) and water (0.6 ml) and ethanol (8 ml) were added. The solution was stirred at 75° C. for 5 hrs. The mixture was concentrated under reduced pressure at 60-75° C. Water (1 ml), ammonia solution (0.5 ml) and acetic anhydride (1 ml) was added to the residue and the mixture stirred at 70-75° C. for 4-5 hrs. The solution was cooled to room temperature, diluted with water (5 ml) and the separated solid filtered. The residue was washed with water (4 ml.) and dried in a vacuum oven at 50° C. to obtain the product as an off-white solid, 0.37 g, in 41% yield.

M.P.: 178-179° C.; MS: 414 (M+1); M.F.: C₁₉H₂₅F₂N₃O₅.

### Method E

To tetrahydrofuran (30 ml) were added triphenylphosphine (2.11 g, 8 mmol)) and diethyldiazocarboxylate (1.62 g, 8 mmol)), and the solution stirred at room temperature. After 10 min acetamide (0.475 g, 8 mmol)) was added and after a further stirring for 10 min, (R)-3-(3,5-difluoro-4-(4-hydroxy-4-(methoxymethyl)piperidin-1-yl)phenyl)-5-(hydroxymethyl) oxazolidin-2-one (2 g, 5.3 mmol) was added and stirring continued further at room temperature. After 16 hrs ice-cold water (4 ml) was added to the reaction mixture and the resulting mixture was extracted by ethyl acetate (2×20 ml). The ethyl acetate extract was dried (over sodium sulfate) and concentrated under reduced pressure. The residue was chromatographed on a column of silica gel to obtain the product as an off-white solid, 0.50 g, yield 22%.

M.P.: 178-179° C.; MS: 414 (M+1); M.F.: C₁₉H₂₅F₂N₃O₅.

### Preparation of Intermediate-11: (S)-N-{3-[3,5-Difluoro-4-(4-methoxymethyl-4-di-O-benzylphosphoryloxy-piperidin-1yl)-phenyl]-2-oxo-oxazolidin-5-ylmethyl}-acetamide

To a solution of (S)-N-{3-[3,5-difluoro-4-(4-methoxymethyl-4-hydroxypiperidine-1yl)-phenyl]-2-oxo-oxazolidin-5-yl methyl}-acetamide (0.2 mmol) and tetrazole (0.6 mmol) in dichloromethane (5 ml) was added dibenzyl N,N,diisopropylphosphoramidite (0.4 mmol) and the resulting mixture was stirred for 4 h. The resulting solution was cooled to 0° C. and 0.6 ml of 0.5M m-chloroperbenzoic acid solution in dichloromethane was added. After 4 h, the solvent was evaporated under residue pressure and the residue chromatographed on a column of silica gel to obtain the product as a off-white solid in 75% yield,

MS: 674 (M+1); M.F. C₃₃H₃₈F₂N₃O₈P;

### Example A

### Phosphoric acid mono-(1-{4-[(S)-5-(acetylamino-methyl)-2-oxo-oxazolidin-3-yl]-2,6-difluorophenyl}-4-methoxymethyl-piperidin-4-yl) ester

To a suspension of (S)-N-{3-[3,5-difluoro-4-(4-methoxymethyl-4-di-O-benzylphosphoryl-oxypiperidine-1yl)phenyl]-2-oxo-oxazolidin-5-yl methyl}-acetamide (0.15 mmol) and 20% palladium hydroxide (20 mg) in 20 ml of a mixture of dichloromethane/aqueous methanol was stirred at room temperature for 6 h. The catalyst was filtered and the residue evaporated under reduced pressure. The residue obtained was triturated with acetone to obtain a white solid as product in 70% yield.

MP. >140° C.; MS: 494(M+1) M.F.: C₁₉H₂₆F₂N₃O₈P.

While the invention has been described in terms of its specific embodiments, certain modifications and equivalents will be apparent to those skilled in the art and are intended to be included within the scope of the invention.

We claim:

1. A Process for the preparation of the Phosphoric acid mono-(1-{4-[(S)-5-(acetylamino-methyl)-2-oxo-oxazolidin-3-yl]-2,6-difluorophenyl}-4-methoxymethyl-piperidin-4-yl)ester of Formula (A), 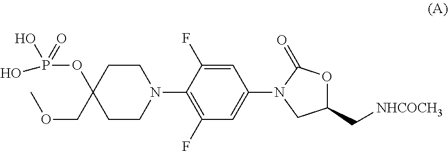
the process comprising the steps of:
a) Converting intermediate of Formula (1) into intermediate of Formula (3) 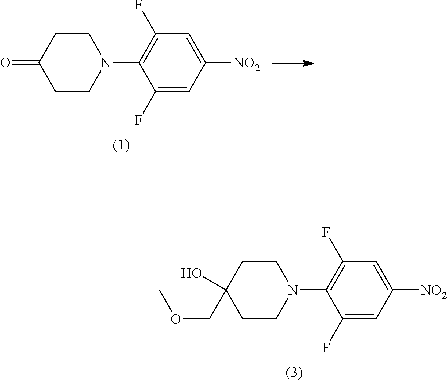 [SMILES: O=C1CCN(c2c(F)cc([N+](=O)[O-])cc2F)CC1 ; COCC1(O)CCN(c2c(F)cc([N+](=O)[O-])cc2F)CC1]
b) Converting intermediate of Formula (3) into intermediate of Formula (5) 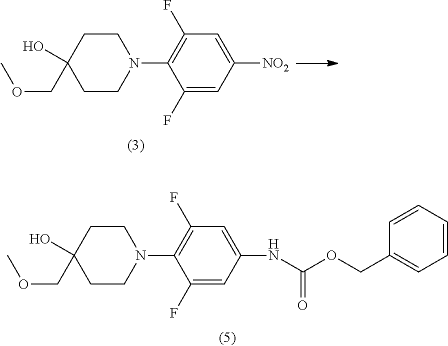 [SMILES: COCC1(O)CCN(c2c(F)cc([N+](=O)[O-])cc2F)CC1 ; COCC1(O)CCN(c2c(F)cc(NC(=O)OCc3ccccc3)cc2F)CC1]
c) Converting intermediate of Formula (5) into intermediate of structure (6) 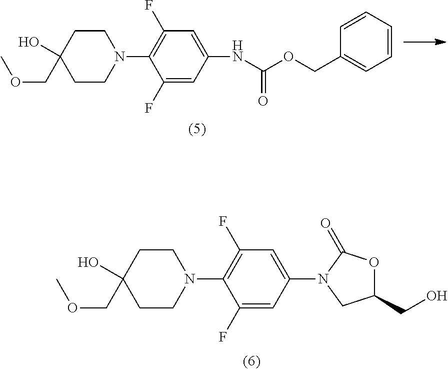 [SMILES: COCC1(O)CCN(c2c(F)cc(NC(=O)OCc3ccccc3)cc2F)CC1 ; COCC1(O)CCN(c2c(F)cc(N3C[C@H](CO)OC3=O)cc2F)CC1]
d) Converting intermediate of Formula (6) into intermediate of Formula (10) 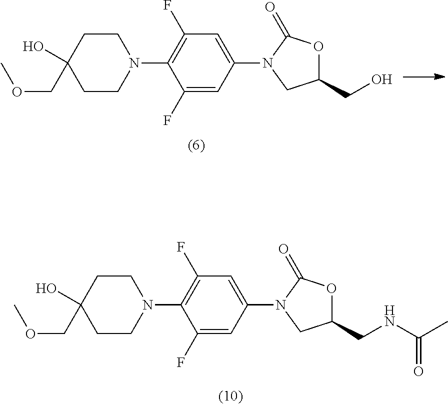 [SMILES: COCC1(O)CCN(c2c(F)cc(N3C[C@H](CO)OC3=O)cc2F)CC1 ; COCC1(O)CCN(c2c(F)cc(N3C[C@H](CNC(C)=O)OC3=O)cc2F)CC1]
e) Converting intermediate of Formula (10) into intermediate of Formula (11), 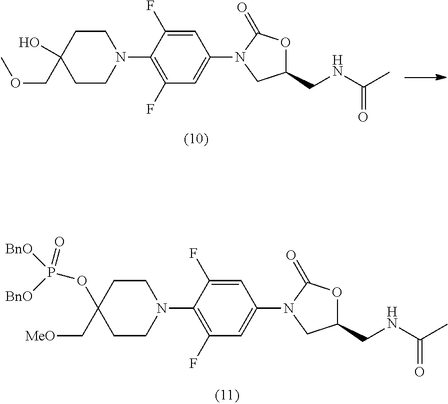 [SMILES: COCC1(O)CCN(c2c(F)cc(N3C[C@H](CNC(C)=O)OC3=O)cc2F)CC1 ; COCC1(OP(=O)(OCc2ccccc2)OCc2ccccc2)CCN(c2c(F)cc(N3C[C@H](CNC(C)=O)OC3=O)cc2F)CC1]
f) Converting intermediate of Formula (11) into compound of Formula (A) or Pharmaceutically acceptable salts thereof

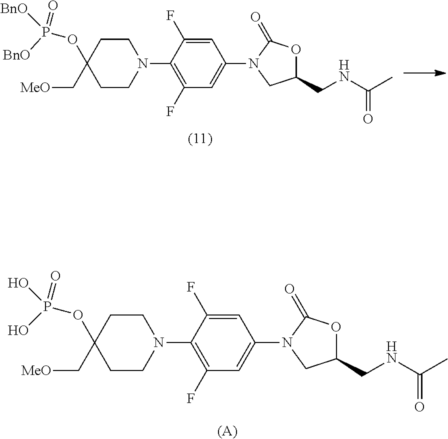

2. The process of claim 1, wherein intermediate of Formula (1) is converted into intermediate of Formula (3) using oxyranylation reagent and alkoxide optionally isolating the intermediate compound of Formula (2).

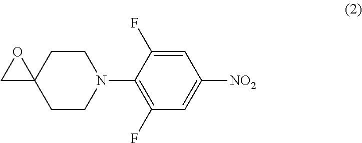 [SMILES: O=[N+]([O-])c1cc(F)c(N2CCC3(CC2)CO3)c(F)c1]

3. The process of claim 1, wherein intermediate of Formula (3) is converted into intermediate of Formula (5) by treating the intermediate of Formula (3) in solvent with reducing agent and benzyl chloroformate solution, optionally isolating the intermediate compound of Formula (4).

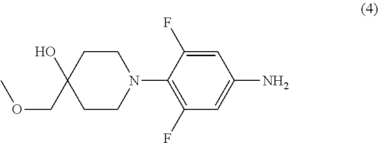 [SMILES: COCC1(O)CCN(c2c(F)cc(N)cc2F)CC1]

4. The process of claim 1, wherein intermediate of Formula (5) is converted into intermediate of Formula (6) by treating intermediate of Formula (5) with R-(−)-glycidyl butyrate in the presence of a base.

5. The process of claim 1, wherein intermediate of Formula (6) is converted into intermediate of Formula (10) by treating intermediate of Formula (6) with mixture of acetamide, triphenylphosphine and an azo compound in presence of an organic solvent.

6. The process of claim 1, wherein intermediate of Formula (6) is converted into intermediate of Formula (10) by the process comprising, converting intermediate of Formula (6) in to wherein intermediate of Formula (8) optionally isolating the compound of Formula (7) and further converting intermediate of Formula (8) in to compound of Formula (10) optionally isolating the compound of Formula (9)

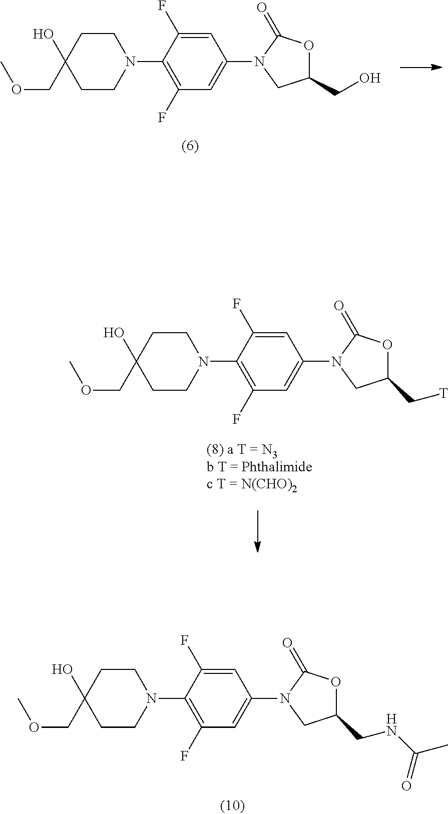

7. The process of claim 6, wherein intermediate of Formula (8) is converted into compound of Formula (10) by treating intermediate of Formula (8) with hydrazine hydrate in an organic solvent.

8. The process of claim 1, wherein compound of Formula (10) is converted into compound of Formula (11) by treating compound of Formula (10) with phosphorylating reagent in the presence of a suitable coupling reagent.

9. The process of claim 1, wherein intermediate of Formula (11) is converted into compound of Formula (A) or pharmaceutically acceptable salts thereof, by treating compound of Formula (11) and a catalyst comprising of 20% palladium hydroxide in a solvent.

10. A compound selected from the group comprising of:
6-(2,6-difluoro-4-nitrophenyl)-1-oxa-6-azaspiro[2.5]octane;
1-(2,6-Difluoro-4-nitro-phenyl)-4-methoxymethyl-piperidin-4-ol;
[3,5-Difluoro-4-(4-hydroxy-4-methoxymethyl-piperidin-1-yl)-phenyl]-carbamic acid benzyl ester;
(5R)-3-[3,5-Difluoro-4-(4-hydroxy-4-methoxymethyl-piperidin-1-yl)-phenyl]-5-hydroxymethyl-oxazolidin-2-one;
(5R)-Methanesulfonic acid 3-[3,5-difluoro-4-(4-hydroxy-4-methoxymethyl-piperidin-1-yl)-phenyl]-2-oxo-oxazolidin-5-ylmethyl ester;
(5R)-3-[3,5-Difluoro-4-(4-hydroxy-4-methoxymethyl-piperidin-1-yl)-phenyl]-5-azidomethyl-oxazolidin-2-one.
